(19) United States
(12) Patent Application Publication
(10) Pub. No.: US 2004/0073292 A1
(43) Pub. Date: Apr. 15, 2004

(54) Vascular reinforcement device and method

(76) Inventors: John M. Adams, Sammamish, WA (US); David G. Reuter, Bothell, WA (US); William James Fitzsimmons, Bellevue, WA (US)

Correspondence Address: Richard O. Gray, Jr., GRAYBEAL JACKSON HALEY LLP, Suite 350, 155 - 108th Avenue NE, Bellevue, WA, 98004-5901, US

(21) Appl. No.: 10/677,592

(22) Filed: Oct. 2, 2003

Related U.S. Application Data
(62) Division of application No. 09/956,623, filed on Sep. 19, 2001, now abandoned.

(51) Int. Cl. A61F 2/06

(52) U.S. Cl. 623/1.15

(57) ABSTRACT

A vascular reinforcement device and method reduces increased vascular pressure of a vein in the presence of forces applied externally to the vein. The device is arranged to continuously overlie of a vein and has a longitudinal dimension and a cross-sectional dimension. The longitudinal dimension is greater than the cross-sectional dimension and is flexible while the cross-sectional dimension of the device is resistant to change. The device is deployed so as to overlie a wall of the vein. The device and method find particular advantageous application for treating preeclampsia or hypertension associated with obesity.

### BACKGROUND OF THE INVENTION

The present invention generally relates to a device and method for reinforcing a vein when exposed to external forces. The present invention more particularly relates to a device and method for reinforcing a renal vein for treating hypertension associated with, for example, obesity or treating preeclampsia.

Preeclampsia is defined as pregnancy induced hypertension associated with either proteinuria (an excess of serum proteins in the urine) and/or edema. It occurs in five to ten percent of pregnancies, typically after the twentieth week of gestation. Risk factors include multiple gestation pregnancies and first time pregnancies.

Preeclampsia can progress to eclampsia, with cerebral symptoms leading to convulsions. The condition is associated with systemic vasospasm wherein arteries throughout the body narrow. This can lead to multi-organ system dysfunction wherein many organs of the body, including the kidneys, brain, eyes, liver, etc., are unable to function normally because of altered blood flow and increased blood pressure.

The cause of preeclampsia is still being debated. However, a new and different cause than that proposed thus far is contemplated by the present invention.

In the human anatomy, the left renal vein, which conducts blood from the left kidney to the inferior vena cava and back to the heart, passes over and is immediately adjacent to the aorta. The aorta is an artery and thus is at high pressure and has a rigid structure when compared to the compliant vascular structure of a relatively low pressure in the left renal vein. The weight and expansion of the abdomen of obese persons or of the pregnant uterus due to the growing fetus in a confined space causes compressive forces to develop within the abdominal area. These forces can compress the compliant left renal vein against the rigid aorta.

Flow through a vein is equal to the pressure drop from one end of the vein to the other divided by the vascular resistance. In a vein, vascular resistance is very sensitive to the diameter of the vein. In fact, vascular resistance in a vein increases in inverse relation to the fourth power of radial decrease. For example, if the radius is halved, the vein pressure increases by a factor of sixteen in order to maintain constant flow. As a further example, and to illustrate how small vein diameter changes can effect vascular pressure, if a vein is normally 6 mm in diameter and the diameter is decreased by 1 mm, the vascular pressure from end to end will double for constant flow.

The kidney among many things tries to maintain homeostasis of body fluid. It is sensitive to fluid pressure and can adjust its vascular resistance over a wide range to maintain relatively constant renal blood flow and filtration rate. As noted above, even a small decrease in the left renal vein diameter can thus result in a tremendous increase in left renal vein vascular pressure as the kidney attempts to maintain a constant blood flow. Hence, the expanding uterus, due to growth of a fetus, can exert external pressure on the left renal vein against the relatively rigid aorta to cause left renal vein diameter decrease and the concomitant extreme increase in left renal vein pressure.

The left kidney will see the increased and higher than normal renal vein pressure. Sensors in the kidney respond to the higher than normal renal vein pressure. In an attempt to increase diuresis the kidney produces a higher volume of renin. Increased renin production leads to increased angiotension II production. Angiotension II causes the blood vessels to constrict, leading to systemic (whole body) vasospasm and increased blood pressure. This in turn leads to aldosterone production which causes water retention in the kidneys. This still in turn causes decreased kidney perfusion due to vasospasm and the vicious cycle continues, known in pregnancy as preeclampsia.

The foregoing events can be triggered by circumstances other than pregnancy. For example, obesity in either men or women can cause external forces to be exerted on the left renal vein against the aorta, leading to a higher than normal renal vein pressure resulting in hypertension and preeclampsia symptoms. Hence, an effective treatment for preeclampsia and hypertension resulting from increased renal vein pressure is urgently needed and desirable. The present invention provides a device and method for such treatment.

### SUMMARY OF THE INVENTION

The present invention provides a method of treating preeclampsia. The method includes the steps of providing a vascular reinforcement device, and placing the vascular reinforcing device adjacent to a wall of the left renal vein in a position which overlies the aorta. The placing step may include the step of implanting the vascular reinforcement device within the left renal vein.

The present invention provides also a method of treating hypertension associated with obesity. The method includes the steps of providing a vascular reinforcement device, and placing the vascular reinforcing device adjacent to a wall of the left renal vein in a position which overlies the aorta. The placing step may include the step of implanting the vascular reinforcement device within the left renal vein.

The present invention further provides a method of reducing increased vascular pressure of a renal vein by forces applied external to the renal vein. The method includes the steps of providing a reinforcement device arranged to continuously overlie, in adjacent relation to, an inner wall of the renal vein, the device having a flexible longitudinal dimension and a relatively rigid cross-sectional dimension resistant to reduction in the presence of applied external forces to the renal vein, and overlying the wall of the renal vein with the reinforcement device.

The present invention still further provides a method of reducing increased vascular pressure of a vein in the presence of forces applied external to the vein. The method includes the steps of providing a reinforcement structure arranged to continuously overlie, in adjacent relation to, a wall of a vein, the reinforcement structure having a longitudinal dimension and a cross-sectional dimension, the longitudinal dimension being greater than the cross-sectional dimension, the reinforcement structure being longitudinally flexible and cross-sectionally resistant to area change and overlying the wall of the vein with the reinforcement structure.

The present invention further provides a vein reinforcement device including reinforcement structure means for lining and reinforcing a wall of the vein, the reinforcement structure means having a longitudinal dimension and a cross-sectional dimension, the longitudinal dimension being greater than the cross-sectional dimension, the longitudinal dimension being flexible and the cross-sectional dimension being resistant to change in the presence of external forces applied to the vein.

The present invention further provides a renal vein reinforcement device comprising a reinforcement structure of substantially cylindrical configuration. The reinforcement structure is arranged to continuously overlie, in adjacent relation to, an inner wall of a renal vein, has a flexible longitudinal dimension and a cross-sectional dimension resistant to reduction in the presence of applied external forces to the renal vein to reduce increased vascular resistance.

The present invention further provides a vein reinforcement device to reduce increased vascular resistance of a vein when exposed to externally applied forces. The device includes a reinforcement structure arranged to continuously overlie, in adjacent relation to, a wall of a vein. The reinforcement structure has a longitudinal dimension and a cross-sectional dimension defining an area, the longitudinal dimension being greater than the cross-sectional dimension, is longitudinally flexible and is cross-sectionally resistant to area change.

### BRIEF DESCRIPTION OF THE DRAWINGS

The features of the present invention which are believed to be novel are set-forth with particularity in the appended claims. The invention, together with further objects and advantages thereof, may best be understood by making reference to the following description in conjunction with the accompanying drawings, in the several figures of which like reference numerals identify identical elements, and wherein:

FIG. 1 is a simplified diagram of the human abdominal cavity illustrating the kidneys, the aorta, the inferior vena cava, the renal veins, and a device embodying the present invention implanted in the left renal vein;

FIG. 2 is a side view of a vein reinforcement device embodying the present invention;

FIG. 3 is a simplified view of a device embodying the present invention being deployed in the left renal vein in accordance with one embodiment of the present invention;

FIG. 4 shows a device embodying the present invention being deployed in the left renal vein prior to expansion of the device in accordance with another embodiment of the present invention;

FIG. 5 is a view, similar to FIG. 4, showing the device after being expanded for deployment in the left renal vein;

FIG. 6 is a side view of another vascular reinforcement device embodying the present invention;

FIG. 7 is an end view of the reinforcement vascular device of FIG. 6; and

FIG. 8 is a side view of a further vein reinforcement device embodying the present invention.

### DETAILED DESCRIPTION OF THE PREFERRED EMBODIMENTS

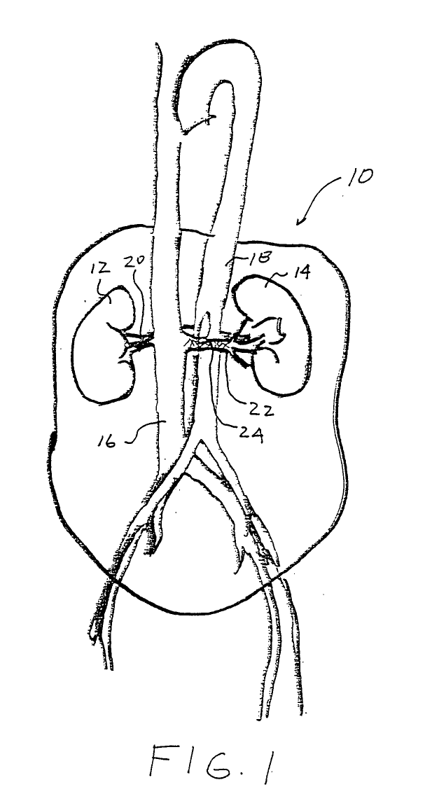

Referring now to FIG. 1, it illustrates an abdominal cavity 10. As illustrated in FIG. 1, the abdominal cavity 10 includes the right kidney 12, a left kidney 14, the inferior vena cava 16, and the aorta 18. Also illustrated in FIG. 1 are the right renal vein 20, the left renal vein 22, and a vascular reinforcement device 24 embodying the present invention. The device 24 is implanted within the left renal vein 22 to reduce increased vascular pressure of the left renal vein when the left renal vein is exposed to external pressure or force.

As previously described, and as may be noted in FIG. 1, the left renal vein 22 is connected between the left kidney 14 and the inferior vena cava 16 to support blood flow from the left kidney 14 to the heart through the inferior vena cava 16. As will also be noted, the left renal vein 22 passes over and adjacent to the aorta 18. The aorta 18 is relatively rigid. Hence, external forces applied to the left renal vein 22 cause the relatively compliant renal vein to deform when pressed against the aorta. This causes a decrease in the left renal vein cross-sectional area or diameter. In an attempt by the left kidney 14 to maintain ample blood flow, the pressure within the left renal vein to the left of the aorta increases. As previously described, the pressure increase can be pronounced for even small decreases in left renal vein size. The increased pressure is sensed by the kidney 14 and causes the kidney to increase the production of renin leading to the previously described cascading effects towards preeclampsia or hypertension.

To reduce such increases in left renal vein pressure, a vascular reinforcement device 24 is implanted within the left renal vein. The device has a cross-sectional area which is resistant to change by the external forces. This allows the device 24 to reinforce the left renal vein against externally applied forces. The result is that the left renal vein pressure increases are reduced in the presence of applied external forces. Excessive-renin production is precluded and preeclampsia or hypertension is avoided.

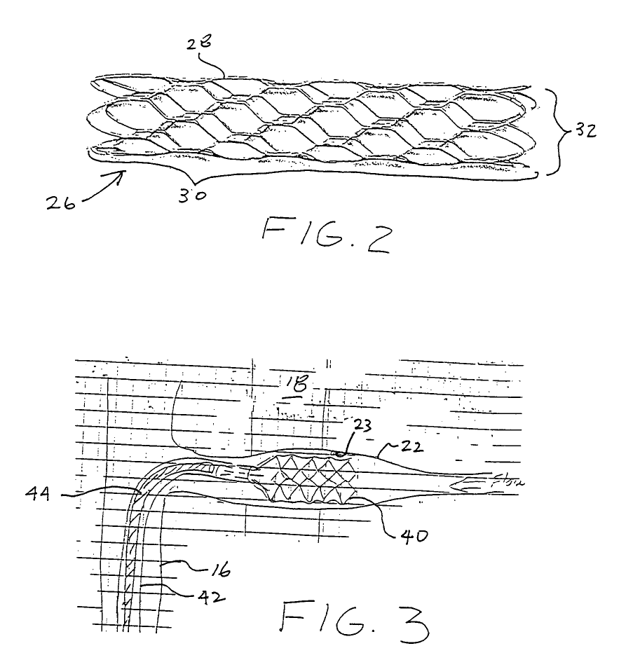

A vascular reinforcement device 26 embodying the present invention is shown in FIG. 2. The device includes a reinforcement structure 28 which may be a laser cut tube of Nitinol, as known in the art, or may be a wire structure formed of Nitinol, for example. Other structures may also be employed such as tubular structures formed of resilient material such as, silicon rubber, for example. The reinforcement structure has a longitudinal dimension 30 at least sufficient for spanning the entire diameter of the aorta and may be long enough to essentially span the entire length of the left renal vein 22. The reinforcement structure 28 also has a cross-sectional area. The cross-sectional area is preferably circular but may have other configurations. The cross-sectional area, in accordance with this embodiment, is defined by a diameter 32.

The device 24 is thus configured to continuously overlie a wall of a vein, such as a left renal vein 22, in close adjacent relation thereto. The device may be dimensioned for overlying an outer wall of the vein. Preferably, the device is dimensioned to overlie an inner wall of a vein as, for example, the device 24 of FIG. 1. To that end, the device may have a diameter on the order of 6 mm for use in the left renal vein, for example. However, as one skilled in the art will appreciate, veins may be of various sizes and hence the device diameter may vary to accommodate different sized veins.

In accordance with the present invention, the device may take advantage of the compliant nature of veins by actually being slightly greater in diameter than the vein in which it is deployed. This provides further assurance that ample vein size will be preserved in the presence of applied external forces.

While the device 26 must be relatively rigid in cross-section in the sense of resisting diameter reduction in the presence of applied external forces, it is still preferably formed of resilient material to be able to return to its original shape should an intense force be applied to it. This avoids the device from being permanently collapsed or crimped by such intense forces to enable the device to remain effective. Hence, to that end, Nitinol is a preferred material. However, other resilient materials known in the art may also be used. In those applications where resilience is of less importance, less resilient materials may be used, such as stainless steel, for example.

Since vascular pressure is generally less than arterial pressure, the device need only resist diameter reduction to external forces of up to about 50 mm of mercury. As used herein the term “resist diameter reduction” is meant to define the ability of the vascular reinforcement device to maintain sufficient cross-sectional area to support blood flow under substantially normal vascular pressure. At the same time, the device must be flexible and compressible to outside forces in excess of 100 mm of mercury, for example. Outside forces may push the vein against arteries near to it and the vein should compress before the artery adjacent to it. Hence, if the device is too resistant to cross-sectional reduction in the presence of external applied forces, it is possible that an underlying artery may be distorted as a result of excessive pressure being exerted against the vein in which the device is employed. Hence, the resilience of the device precludes such distortion of adjacent arteries and thus prevents constriction of adjacent arteries. However, since the device is resilient, once the excessive force subsides, the device will return to its original shape to maintain a low vascular pressure within the vein in which it is deployed.

As illustrated in FIG. 2, the longitudinal dimension 30 is greater than the cross-sectional dimension defined by the diameter 32. The device 26 is longitudinally flexible. This permits the device to comply to the shape of adjacent rigid body structures such as the aorta or other arteries.

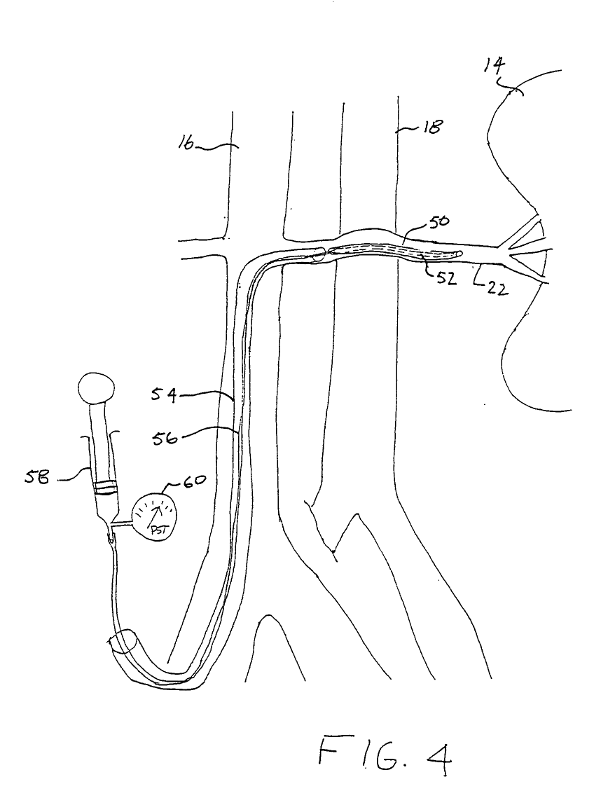

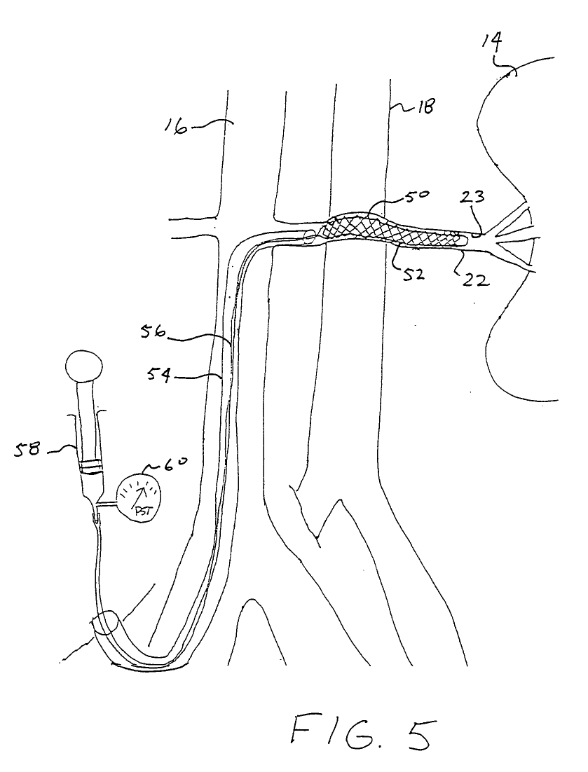

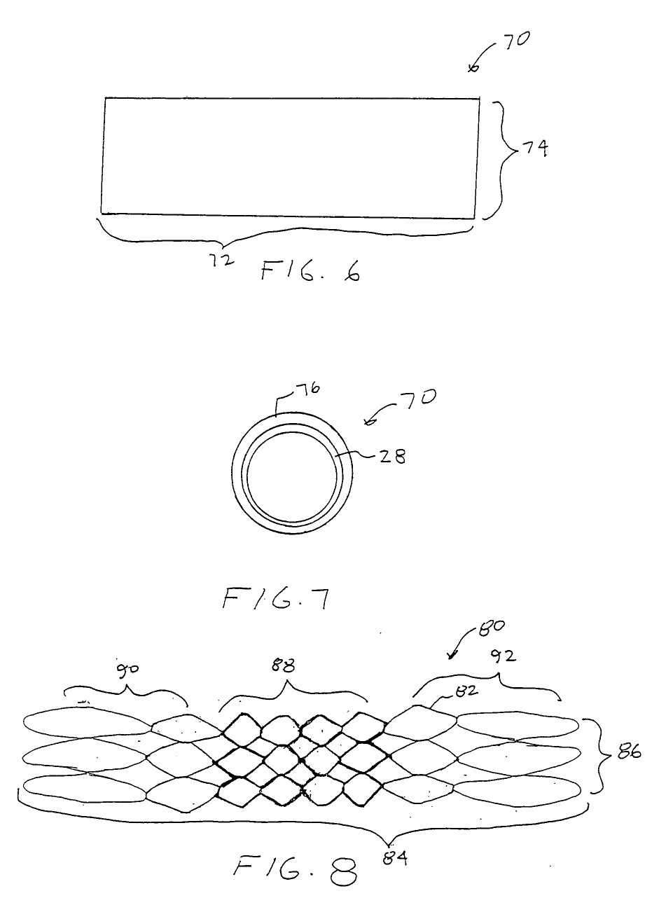

FIG. 3 shows another vascular reinforcement device 40 embodying the present invention being implanted in the left renal vein 22. As shown in FIG. 3, the device is implanted by first advancing a catheter 42 up a vein of the leg, such as the femoral vein, (not shown) into the inferior vena cava 16. The catheter is advances into the left renal vein 22 as illustrated. The device 40, which is expandable, is then advanced through the catheter 42 in a collapsed state by a push tube 44. When the device 40 reaches the left renal vein where it crosses the aorta 18, the device is released by further advancement, whereupon it expands to continuously line an inner wall 23 of the left renal vein 22. After the device 40 is thus deployed, the advancement tube 44 and catheter 42 are removed.

FIG. 4 shows another vascular reinforcement device 50 embodying the present invention being employed in accordance with a further embodiment. Here, the device 50 is balloon expandable by a balloon 52.

The device 50 is implanted by first advancing a catheter 54 into the inferior vena cava 16 and into the left renal vein 22 as illustrated. Next, the device 50 and balloon 52 are advanced by a flow tube 56 through the catheter 54 and into the left renal vein 22 where the left renal vein 22 crosses the aorta 18. Then, a pressure device 58 having a pressure meter 60 is used to inflate the balloon 52.

As best seen in FIG. 5, the inflated balloon 52 has expanded the device 50 to continuously line the inner wall 23 of the left renal vein 22. Once the device is fully expanded, the balloon is deflated. The deflated balloon 52, flow tube 56, and catheter 54 are then removed leaving the device 50 in place to reinforce the left renal vein and reduce increased vascular pressure within the left renal vein notwithstanding applied external forces to the left renal vein.

FIGS. 6 and 7 show another vascular reinforcement device 70 embodying the present invention. The device 70 is generally cylindrical in configuration having a longitudinal dimension 72 and a cross-sectional dimension defined by a diameter 74. As may best be seen in FIG. 7, the device 70 includes the reinforcement structure 28 previously illustrated in FIG. 2. Here, however, the reinforcement structure 28 is coated or covered with an external coating 76. The external coating may be formed of silicon rubber, Teflon, or polyurethane. The coating 72 is made thin enough for flexibility so as to enable the device 70 to retain all of the structure benefits of the reinforcement structure 28 as previously described.

A further vascular reinforcement device 80 embodying the present invention is shown in FIG. 8. The device includes a reinforcement structure 82 which again may be a wire structure formed of Nitinol, for example, or a laser cut Nitinol tube. The reinforcement structure has a longitudinal dimension 84 at least sufficient for spanning the entire diameter of the aorta and may be long enough to essentially span the entire length of the left renal vein 22. The reinforcement structure 82 also has a cross-sectional area. The cross-sectional area is preferably circular but may have other configurations. The cross-sectional area, in accordance with this embodiment, is defined by a diameter 86.

In addition to the structural features of the vein reinforcement devices previously described, the device 80 offers a further feature. Here it may be noted that the midsection 88 of the device 80 is formed of wire which is heavier stock than the wire forming end sections 90 and 92. Preferably, the wire stock gradually decreases in diameter from the midsection 88 to the end sections 90 and 92. When the device 80 is formed by laser cutting a Nitinol tube, the midsection 88 may have supports which are wider than the supports in the end sections 90 and 92. This allows the device 88 to become gradually soft out from the device midsection to provide a transition zone. This may prevent trauma to the vein when external forces are present. More particularly, the potential for the vein to shear at the end of the device when exposed to external forces is reduced while the midsection still provides reduced increases in vascular pressure. Hence, the device offers less change in cross-sectional area from the midsection 88 to the end sections 90 and 92 while the midsection 88 offers sufficient resistance to cross-sectional change to permit the device to sufficiently reduce increases in vascular pressure.

While particular embodiments of the present invention have been shown and described, modifications may be made, and it is therefore intended in the impended claims to cover all such changes and modifications which fall within the true spirit and scope of the invention.

What is claimed is:

1. A vein reinforcement device to reduce increased vascular resistance of a vein when exposed to externally applied forces, the device including a reinforcement structure arranged to continuously overlie, in adjacent relation to, a wall of a vein, the reinforcement structure having a longitudinal dimension and a cross-sectional dimension defining an area, the longitudinal dimension being greater than the cross-sectional dimension, the reinforcement structure being longitudinally flexible and cross-sectionally resistant to area change.

2. The device of claim 1 wherein the cross-sectional dimension is substantially circular.

3. The device of claim 1 wherein the reinforcement structure is formed of a metal.

4. The device of claim 3 wherein the reinforcement structure is formed of one of stainless steel and Nitinol.

5. The device of claim 3 wherein the reinforcement structure is formed of a wire structure.

6. The device of claim 5 wherein the reinforcement structure further includes an external coating overlying the wire structure.

7. The device of claim 6 wherein the external coating is formed of one of silicone rubber and Teflon.

8. The device of claim 1 wherein the cross-sectional dimension configures the reinforcement structure for overlying the vein wall externally to the vein.

9. The device of claim 1 wherein the cross-sectional dimension configures the reinforcement structure for overlying the vein wall within the vein.

10. The device of claim 9 wherein the reinforcement structure is an expandable structure.

11. The device of claim 1 wherein the reinforcement structure is only cross-sectionally resistant to area change for applied forces less than about 50 mm Hg.

12. A renal vein reinforcement device comprising a reinforcement structure of substantially cylindrical configuration, the reinforcement structure arranged to continuously overlie, in adjacent relation to, an inner wall of a renal vein, having a flexible longitudinal dimension and a cross-sectional dimension resistant to reduction in the presence of applied external forces to the renal vein to reduce increased vascular resistance.

13. The device of claim 12 wherein the reinforcement structure is formed of a metal.

14. The device of claim 13 wherein the reinforcement structure is formed of one of stainless steel and Nitinol.

15. The device of claim 13 wherein the reinforcement structure is formed of a wire structure.

16. The device of claim 15 wherein the reinforcement structure further includes an external coating overlying the wire structure.

17. The device of claim 16 wherein the external coating is formed of one of silicone rubber and Teflon.

18. The device of claim 12 wherein the reinforcement structure is an expandable structure to permit the reinforcement structure to be positioned within the renal vein in a collapsed state and thereafter expanded to the substantially cylindrical configuration.

19. The device of claim 12 wherein the reinforcement structure has a longitudinal center axis and wherein the cross-sectional dimension is only resistant to reduction in the presence of applied radial pressure of less than about 50 mm Hg.

20. A vein reinforcement device, the device including reinforcement structure means for lining and reinforcing a wall of the vein, the reinforcement structure means having a longitudinal dimension and a cross-sectional dimension, the longitudinal dimension being greater than the cross-sectional dimension, the longitudinal dimension being flexible and the cross-sectional dimension being resistant to change in the presence of external forces applied to the vein.

21. The device of claim 20 wherein the cross-sectional dimension is substantially circular.

22. The device of claim 20 wherein the reinforcement structure means is formed of a metal.

23. The device of claim 22 wherein the reinforcement structure means is formed of one of stainless steel and Nitinol.

24. The device of claim 22 wherein the reinforcement structure means is formed of a wire structure.

25. The device of claim 24 wherein the reinforcement structure means further includes an external coating overlying the wire structure.

26. The device of claim 25 wherein the external coating is formed of one of silicone rubber and Teflon.

27. The device of claim 20 wherein the cross-sectional dimension configures the reinforcement structure means for lining an external wall of the vein.

28. The device of claim 20 wherein the cross-sectional dimension configures the reinforcement structure means for lining an inner wall of the vein.

29. The device of claim 26 wherein the reinforcement structure means is expandable from a collapsed condition to a deployed expanded condition.

30. The device of claim 20 wherein the reinforcement structure means has a longitudinal center axis and wherein the cross-sectional dimension is only resistant to reduction in the presence of forces of less than about 50 mm Hg.

31. A method of reducing increased vascular pressure in a vein in the presence of forces applied external to the vein, the method including the steps of:
providing a reinforcement structure arranged to continuously overlie, and adjacent relation to, a wall of a vein, the reinforcement structure having a longitudinal dimension and a cross-sectional dimension, the longitudinal dimension being greater than the cross-sectional dimension, the reinforcement structure being longitudinally flexible and cross-sectionally resistant to area change; and
overlying the wall of the vein with the reinforcement structure.

32. The method of claim 31 wherein the overlying step includes the step of overlying an outer wall of the vein with the reinforcement structure.

33. The method of claim 31 wherein the overlying step includes the step of overlying an inner wall of the vein with the reinforcement structure.

34. The method of claim 33 further including the step of guiding the reinforcement structure into position within the vein through a catheter.

35. The method of claim 33 wherein the reinforcement structure is initially in a collapsed state and expandable to a deployed state and wherein the method further includes the steps of positioning the reinforcement structure within the vein while the reinforcement structure is in the collapsed state and thereafter expanding the reinforcement structure to the deployed state.

36. The method of claim 35 wherein the expanding step includes the step of expanding the reinforcement structure with a balloon.

37. The method of claim 35 further including the steps of feeding a catheter having a distal end into the vein until the distal end is at a desired position within the vein and thereafter, advancing the reinforcement structure through the catheter to the desired position.

38. A method of reducing increased vascular pressure in a renal vein in the presence of forces applied external to the renal vein, the method including the steps of:
providing a reinforcement device arranged to continuously overlie, in adjacent relation to, an inner wall of the renal vein, the device having a flexible longitudinal dimension and a relatively rigid cross-sectional dimension resistant to reduction in the presence of applied external forces to the renal vein; and
overlying the wall of the renal vein with the reinforcement device.

39. The method of claim 38 wherein the overlying step includes the step of overlying an outer wall of the renal vein with the reinforcement device.

40. The method of claim 38 wherein the overlie step includes the step of overlying an inner wall of the renal vein with the reinforcement device.

41. The method of claim 40 further including the step of guiding the reinforcement device into position through a catheter.

42. The method of claim 40 wherein the reinforcement device is initially in a collapsed state and expandable to a deployed state and wherein the method further includes the steps of positioning the reinforcement device within the renal vein while the reinforcement device is in the collapsed state and thereafter expanding the reinforcement device to the deployed state.

43. The method of claim 42 wherein the expanding step includes the step of expanding the reinforcement device with a balloon.

44. The method of claim 42 further including the steps of feeding a catheter having a distal end into the vein until the distal end is at a desired position within the vein and thereafter, advancing the reinforcement device through the catheter to the desired position.

45. The method of claim 38 wherein the renal vein is the left renal vein and wherein the overlying steps includes the step of overlying the wall of the renal vein with the reinforcement device at a desired position where the left renal vein crosses the aorta.

46. The method of claim 45 wherein the overlying step includes the step of overlying an inner wall of the left renal vein with the reinforcement device.

47. The method of claim 46 further including the step of guiding the reinforcement device to the desired position through a catheter.

48. The method of claim 46 wherein the reinforcement device is initially in a collapsed state and expandable to a deployed state and wherein the method further includes the steps of positioning the reinforcement device within the left renal vein while the reinforcement device is in the collapsed state and thereafter expanding the reinforcement device to the deployed state.

49. The method of claim 48 wherein the expanding step includes the step of expanding the reinforcement device with a balloon.

50. The method of claim 48 further including the steps of feeding a catheter having a distal end into the left renal vein until the distal end is at the desired position within the left renal vein and thereafter, advancing the reinforcement device through the catheter to the desired position.

51. A method of treating preeclampsia, the method including the steps of:
providing a vascular reinforcement device; and
placing the vascular reinforcing device adjacent to a wall of the left renal vein in a position which overlies the aorta.

52. The method of claim 51 wherein the placing step includes implanting the vascular reinforcement device within the left renal vein.

53. The method of claim 52 wherein the implanting step includes the step of guiding the vascular reinforcement device into position with the left renal vein through a catheter.

54. The method of claim 52 wherein the vascular reinforcement device is initially in a collapsed state and expandable to a deployed state and wherein the method further includes the steps of positioning the vascular reinforcement device within the vein while the vascular reinforcement device is in the collapsed state and thereafter expanding the vascular reinforcement device to the deployed state.

55. The method of claim 54 wherein the expanding step includes the step of expanding the vascular reinforcement device with a balloon.

56. The method of claim 54 further including the steps of feeding a catheter having a distal end into the left renal vein and advancing the vascular reinforcement device, while in the collapsed state, through the catheter into position within the left renal vein.

57. A method of treating hypertension associated with obesity, the method including the steps of:
providing a vascular reinforcement device; and
placing the vascular reinforcing device adjacent to a wall of the left renal vein in a position which overlies the aorta.

58. The method of claim 57 wherein the placing step includes implanting the vascular reinforcement device within the left renal vein.

59. The method of claim 58 wherein the implanting step includes the step of guiding the vascular reinforcement device into position within the left renal vein through a catheter.

60. The method of claim 58 wherein the vascular reinforcement device is initially in a collapsed state and expandable to a deployed state and wherein the method further includes the steps of positioning the vascular reinforcement device within the vein while the vascular reinforcement device is in the collapsed state and thereafter expanding the vascular reinforcement device to the deployed state.

61. The method of claim 60 wherein the expanding step includes the step of expanding the vascular reinforcement device with a balloon.

62. The method of claim 60 further including the steps of feeding a catheter having a distal end into the left renal vein and advancing the vascular reinforcement device, while in the collapsed state, through the catheter into position within the left renal vein.

63. The device of claim 1 wherein the reinforcement structure includes a midsection and a pair of end sections and wherein the midsection is configured to provide a greater resistance to cross-sectional area change than the end sections.

64. The device of claim 63 wherein the reinforcement structure is a wire structure.

65. The device of claim 64 wherein the wire structure includes heavier wire stock in the midsection than in the end sections.
